SEQUENCE LISTING (3 sequences)

SEQ ID NO: 1 - RNA, 1857 nt, artificial
  R491 RNA
gggagaaagcuugaggauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccguguaagacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaauauuccccccccccccccccccccccccccccccucuagacaauuggaauu

SEQ ID NO: 2 - RNA, 1870 nt, artificial
  R1265 RNA
gggagaaagcuugaggauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccguguaagacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 3 - RNA, 2035 nt, artificial
  R2244 RNA
ggggcgcugccuacggagguggcagccaucuccuucucggcaucaagcuugaggauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccguguaagacuagugcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccuagaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

(19) United States
(12) Patent Application Publication
(10) Pub. No.: US 2019/0049414 A1
(43) Pub. Date: Feb. 14, 2019

(54) METHOD FOR ANALYZING BY-PRODUCTS OF RNA IN VITRO TRANSCRIPTION

(71) Applicant: CureVac AG, Tübingen (DE)

(72) Inventors: Aniela WOCHNER, Tübingen (DE); Isabel REICHERT, Nürtingen-Oberensingen (DE); Kiriaki RAPTOPOULOU, Tübingen (DE)

(21) Appl. No.: 16/077,973

(22) PCT Filed: Feb. 15, 2016
(86) PCT No.: PCT/EP2016/053194

§ 371 Date: Aug. 14, 2018

(51) Int. Cl. G01N 30/34; B01D 15/32; B01D 15/36

(52) U.S. Cl. CPC G01N 30/34; G01N 2030/8827; B01D 15/366; B01D 15/325

(57) ABSTRACT

The present invention relates to the detection and analysis of by-products in a process of RNA in vitro transcription by HPLC. It further relates to the use of this method for the quality control of RNA produced by in vitro transcription or for identifying suitable RNA purification conditions.

### FIELD OF THE INVENTION

The present invention relates to the detection and analysis of by-products in a process of RNA in vitro transcription by HPLC. It further relates to the use of this method for the quality control of RNA produced by in vitro transcription or for identifying suitable RNA purification conditions.

### BACKGROUND OF THE INVENTION

For the therapeutic use of RNA in patients, a rigorous quality control of the RNAs to be used is mandatory. An important issue is the determination of RNA purity. Apart from RNA integrity, which is commonly determined via gel electrophoresis, limited knowledge is available as to which parameters are important for RNA quality.

During transcription, RNAs shorter than the target RNA are also produced by the polymerase. These may alter the properties of the mRNA product, not only in terms of concentration, but also in terms of biological activity, if not thoroughly removed.

It is well established that RNA transcribed in vitro by phage polymerase contains multiple aberrant RNAs, including short RNAs as a result of abortive transcription initiation events (Milligan et al. (1987) Nucl. Acids Res 15:8783-8798) and double stranded (ds)RNAs generated by RNA dependent RNA polymerase activity (Arnaud-Barbe et al. (1998) Nucl. Acids Res 26:3550-3554; Nacheva and Berzal-Herranz (2003) Eur. J. Biochem. 270:1458-1465).

Kariko et al. identified that these contaminants from in vitro transcribed RNA are a source of innate immune activation and that their removal increases RNA translation and eliminates type I interferon and inflammatory cytokine secretion (Karikó K. et al. (2011) Nucl. Acids Res. 39(21):e142)

These short RNAs cannot be detected in standard gel electrophoresis analysis of long mRNAs wherein RNA is visualized by intercalating dyes such as ethidium bromide. These dyes intercalate and/or interact with the phosphate backbone, resulting in good visualization of longer nucleic acids, whereas short RNAs are difficult to detect, especially when present in a mixture with longer RNAs, such as mRNAs. Additionally, the methods used to resolve long RNAs cannot be used to resolve short RNAs.

For the future development of RNA products/medicaments it is mandatory to develop a method for determining the presence and quantity of short RNA by-products as a quality control.

WO 2015/101416 A1 and PCT/EP2015/001336 describe methods for analyzing an RNA molecule wherein the RNA molecule is cleaved with a catalytic nucleic acid molecule and the resulting RNA fragments are analyzed.

WO 2014/144039 A1 describes a method for characterizing an RNA transcript using a procedure selected from the group consisting of oligonucleotide mapping, reverse transcriptase sequencing, charge distribution analysis and detection of RNA impurities.

Hence, the problem of the invention is to provide a sensitive method for reliably detecting by-products of RNA in vitro transcription.

### SUMMARY OF THE INVENTION

The present inventors have surprisingly found that short by-products of RNA in vitro transcription can be detected and analyzed by HPLC. An HPLC protocol has been developed that allows single-nucleotide resolution of RNA oligomers for monitoring and analysis of transcription reactions and RNA products. Using this protocol, contamination of the RNA product with by-products can be determined and quantified. Additionally, fraction collection of selected peaks during HPLC purification allows the isolation and subsequent characterization of the RNA species comprised in the by-products for identifying crucial sequence motifs responsible for the generation of by-products and for identifying better purification methods to improve RNA quality.

Accordingly, the present invention relates to a method for detecting by-products of in vitro transcription in a sample comprising an in vitro transcribed target RNA, the method comprising the steps of:
a) preparing a sample comprising a target RNA by in vitro transcription;
b) purifying the target RNA, thereby providing a purified target RNA sample;
c) detecting the by-products in the purified target RNA sample by HPLC.

Preferably, the method does not comprise a step of treating the target RNA with a ribozyme.

The by-products may comprise at least two nucleic acid molecules with different length and may have a length of 5 to 500 nucleotides.

Preferably, the by-products do not comprise the 3′ terminus of the target RNA. The by-products may be homooligomers of nucleotides, short single-stranded RNAs, double-stranded RNAs and/or DNA-RNA hybrids.

Step b) may be performed under denaturing conditions and/or may comprise a step of purifying the target RNA by HPLC, which preferably is reversed-phase HPLC.

In the HPLC a porous reversed phase may be used as stationary phase, which preferably is a porous, non-alkylated polystyrene/divinylbenzene matrix.

The HPLC in step c) may be ion-pair, reversed-phase HPLC and/or may use a carbon-chain bonded silica column.

Preferably, the carbon-chain bonded silica column is an octadecyl carbon chain (C18)-bonded silica column.

The silica column may be prepared from tetraethoxysilane and bis(triethoxysilyl)ethane which may be used in a 4:1 mole ratio.

In one embodiment the column has a particle size of 0.5 to 5 μm and/or has a pore size of 50 to 300 Å.

The HPLC of step c) may use a mixture of an aqueous solvent and an organic solvent as mobile phase.

The aqueous solvent may be a buffer which may be selected from the group consisting of triethylammonium acetate, trifluoroacetic acid, acetic acid, formic acid, acetate buffer, phosphate buffer, tetrabutylammonium bisulfate, tetrabutylammonium bromide and tetrabutylammonium chloride.

Preferably, the buffer is a 0.1 M triethylammonium acetate buffer.

The organic solvent may be selected from the group consisting of acetonitrile, methanol, ethanol, 1-propanol, 2-propanol, hexafluoroisopropanol, acetone and a mixture thereof and preferably it may be acetonitrile.

In one embodiment the mobile phase contains 3 to 5% organic solvent, relative to the mobile phase, the rest being the aqueous solvent at the beginning of the HPLC process.

In one embodiment a gradient separation proceeds and preferably the proportion of organic solvent is increased to provide the gradient. More preferably, the proportion of organic solvent in the mobile phase is increased in the course of HPLC separation from 3.5% to 100%.

The method may further comprise a step d) of isolating and/or characterizing the by-products.

The by-products may be characterized by enzyme assays, mass spectrometry and/or sequencing.

In one embodiment the amount of the by-products relative to the total amount of RNA is determined.

The method of the present invention may be used to identify sequence motifs within the target RNA which are responsible for the generation of by-products.

The method of the present invention may also be used for the quality control of RNA produced by in vitro transcription.

The method of the present invention may also be used to identify suitable RNA purification conditions.

The method of the present invention may also be used to compare RNA purification conditions.

### BRIEF DESCRIPTION OF THE DRAWINGS

FIG. 1: By-products of in vitro transcription of PpLuc mRNA (R491 (light grey), R1265 (dark grey), and R2244 (black)) detected via analytical HPLC (C18 column).

(a) Chromatogram for entire analytical run. Full-length mRNA elutes at around 24 min.
(b) Enlargement of the area between 1 and 23 min of the chromatogram of (a). Short RNAs are resolved by single nucleotides. Three distinct populations of short RNAs have been identified:
“early peaks” (1), whose generation is dependent upon the mRNA start sequence (occurring only in R2244) and which consist of guanosine multimers (as determined by mass spectrometry);
“middle peaks” (2), which are antisense RNA oligomers transcribed via RNA-dependent RNA polymerization (as determined by mass spectrometry), in this case from the open reading frame sequence of PpLuc;
“late peaks” (3).

FIG. 2: HPLC analysis of PpLuc mRNA (R491 (light grey), R1265 (dark grey), and R2244 (black)) previously purified via an improved preparative HPLC method. Hybridized RNA oligomers could be removed, resulting in high quality RNA.

(a) Chromatogram for entire analytical run. Full-length mRNA elutes at around 24 min.
(b) Enlargement of the area between 1 and 23 min of the chromatogram of (a) (same scaling as in FIG. (1b)).

### DEFINITIONS

For the sake of clarity and readability the following definitions are provided. Any technical feature mentioned in these definitions may be read on each and every embodiment of the invention. Additional definitions and explanations may be specifically provided in the context of these embodiments.

In vitro transcription: The term “in vitro transcription” relates to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which according to the present invention is preferably a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In a preferred embodiment of the present invention the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Methods for in vitro transcription are known in the art (Geall et al. (2013) Semin. Immunol. 25(2): 152-159; Brunelle et al. (2013) Methods Enzymol. 530:101-14). Reagents used in said method typically include:

1) a linearized DNA template with a promoter sequence that has a high binding affinity for its respective RNA polymerase such as bacteriophage-encoded RNA polymerases;
2) ribonucleoside triphosphates (NTPs) for the four bases (adenine, cytosine, guanine and uracil);
3) optionally a cap analog as defined below (e.g. m7G(5′)ppp(5′)G (m7G));
4) a DNA-dependent RNA polymerase capable of binding to the promoter sequence within the linearized DNA template (e.g. T7, T3 or SP6 RNA polymerase);
5) optionally a ribonuclease (RNase) inhibitor to inactivate any contaminating RNase;
6) optionally a pyrophosphatase to degrade pyrophosphate, which may inhibit transcription;
7) MgCl₂, which supplies Mg²⁺ ions as a co-factor for the polymerase;
8) a buffer to maintain a suitable pH value, which can also contain antioxidants (e.g. DTT), and/or polyamines such as spermidine at optimal concentrations.

By-product: A by-product is a secondary product of a manufacturing process or a chemical reaction, which differs from the target product of said process or reaction in its size and/or chemical structure. Within the present invention the by-product is produced by the RNA polymerase during the RNA in vitro transcription process.

Within the present invention the by-product may comprise homooligo- or -polymers of a nucleotide, such as oligomers of guanosine, for example oligomers comprising 3 to 10 guanosine nucleotides.

Additionally or alternatively, the by-product may comprise short RNAs which have a lower number of nucleotides than the target RNA, but have part of the sequence of the target RNA and may therefore also be considered as fragments of the target RNA. These short RNAs may for example be produced by premature termination of transcription, i.e. the transcription stops before the end of the sequence to be transcribed is reached. Hence, these short RNAs typically comprise the 5′ sequence of the target RNA.

Also additionally or alternatively, the by-product may comprise long RNAs which have a higher number of nucleotides than the target RNA and comprise the complete sequence of the target RNA and additional nucleotides. These long RNAs may for example be produced by incomplete termination of the transcription or by incomplete linearization of the plasmid providing the template DNA.

Also additionally or alternatively, the by-products may comprise double-stranded RNA or DNA/RNA hybrids which are produced by RNA-dependent polymerization catalyzed by the RNA polymerase. In the method of the present invention the detection of antisense RNA or DNA molecules may be indicative for these by-products.

The by-product may also be an RNA having the same or a shorter or longer length as the target RNA in which one or more modified nucleotides are present, if the target RNA does not comprise modified nucleotides.

Nucleic acid: The term nucleic acid means any DNA- or RNA-molecule and is used synonymously with polynucleotide. Furthermore, modifications or derivatives of the nucleic acid as defined herein are explicitly included in the general term “nucleic acid”. For example, peptide nucleic acid (PNA) is also included in the term “nucleic acid”.

Target RNA: The target RNA is the RNA which is to be produced by the RNA in vitro transcription process. The length and the sequence of the target RNA is determined by the sequence of the nucleic acid template which is subjected to the RNA in vitro transcription reaction. Hence, the target RNA is the full-length RNA transcript. In contrast, the by-products typically are either longer or shorter than the target RNA. The target RNA may further comprise a cap structure on its 5′ terminus, if a cap analog is added to the RNA in vitro transcription reaction. The target RNA may also comprise modified nucleotides, if these modified nucleotides had been added to the RNA in vitro transcription reaction mixture. In contrast, RNA containing modified nucleotides which had not been added to the RNA in vitro transcription reaction mixture is considered as a by-product.

If the target RNA is mRNA, it will preferably code for proteins, in particular those which have an antigen character, and for example all recombinantly produced or naturally occurring proteins, which are known to a person skilled in the art from the prior art and are used for therapeutic, diagnostic or research purposes. In particular, the antigens may be tumour antigens or antigens of pathogens, for example of viral, bacterial or protozoal organisms.

RNA, mRNA: RNA is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. Usually RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, an open reading frame, optionally a 3′UTR and a poly(A) sequence.

In addition to messenger RNA, several non-coding types of RNA exist which may be involved in regulation of transcription and/or translation, and immunostimulation.

The term “RNA” further encompasses RNA molecules, such as viral RNA, retroviral RNA and replicon RNA, small interfering RNA (siRNA), antisense RNA, CRISPR RNA, ribozymes, aptamers, riboswitches, immunostimulating RNA, transfer RNA (tRNA), ribosomal RNA (rRNA), small nuclear RNA (snRNA), small nucleolar RNA (snoRNA), microRNA (miRNA), and Piwi-interacting RNA (piRNA).

Modified nucleoside triphosphate: The term “modified nucleoside triphosphate” as used herein refers to chemical modifications comprising backbone modifications as well as sugar modifications or base modifications. These modified nucleoside triphosphates are herein also called (nucleotide) analogs.

In this context, the modified nucleoside triphosphates as defined herein are nucleotide analogs/modifications, e.g. backbone modifications, sugar modifications or base modifications. A backbone modification in connection with the present invention is a modification, in which phosphates of the backbone of the nucleotides are chemically modified. A sugar modification in connection with the present invention is a chemical modification of the sugar of the nucleotides. Furthermore, a base modification in connection with the present invention is a chemical modification of the base moiety of the nucleotides. In this context nucleotide analogs or modifications are preferably selected from nucleotide analogs which are applicable for transcription and/or translation.

### Sugar Modifications

The modified nucleosides and nucleotides, which may be used in the context of the present invention, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

The sugar group can also contain one or more carbons that possess the opposite stereochemical configuration than that of the corresponding carbon in ribose. Thus, a modified nucleotide can include nucleotides containing, for instance, arabinose as the sugar.

### Backbone Modifications

The phosphate backbone may further be modified in the modified nucleosides and nucleotides. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

### Base Modifications

The modified nucleosides and nucleotides, which may be used in the present invention, can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In some embodiments, the nucleotide analogs/modifications are selected from base modifications, which are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, l-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group.

In specific embodiments, a modified nucleoside is 5′-O-(1-Thiophosphate)-Adenosine, 5 ‘-O-(1-Thiophosphate)-Cytidine, 5’-O-(1-Thiophosphate)-Guanosine, 5′-O-(1-Thiophosphate)-Uridine or 5′-O-(1-Thiophosphate)-Pseudouridine.

In further specific embodiments the modified nucleotides include nucleoside modifications selected from 6-aza-cytidine, 2-thio-cytidine, α-thio-cytidine, Pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, Pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, α-thio-adenosine, 8-azido-adenosine, 7-deaza-adenosine.

Further modified nucleotides have been described previously (WO 2013/052523).

5′-Cap structure: A 5′ cap is typically a modified nucleotide, particularly a guanine nucleotide, added to the 5′ end of an RNA molecule. Preferably, the 5′ cap is added using a 5′-5′-triphosphate linkage. A 5′ cap may be methylated, e.g. m7GpppN, wherein N is the terminal 5′ nucleotide of the nucleic acid carrying the 5′ cap, typically the 5′-end of an RNA. The naturally occurring 5′ cap is m7GpppN.

Further examples of 5′ cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

Particularly preferred 5′ cap structures are CAP1 (methylation of the ribose of the adjacent nucleotide of m7G), CAP2 (methylation of the ribose of the 2^{nd} nucleotide downstream of the m7G), CAP3 (methylation of the ribose of the 3^{rd} nucleotide downstream of the m7G) and CAP4 (methylation of the ribose of the 4^{th} nucleotide downstream of the m7G).

A 5′ cap structure may be formed by a cap analog.

Cap analog: A cap analog refers to a non-extendable di-nucleotide that has cap functionality which means that it facilitates translation or localization, and/or prevents degradation of the RNA molecule when incorporated at the 5′ end of the RNA molecule. Non-extendable means that the cap analog will be incorporated only at the 5′terminus because it does not have a 5′ triphosphate and therefore cannot be extended in the 3′ direction by a template-dependent RNA polymerase.

Cap analogs include, but are not limited to, a chemical structure selected from the group consisting of m7GpppG, m7GpppA, m7GpppC; unmethylated cap analogs (e.g., GpppG); dimethylated cap analog (e.g., m2,7GpppG), trimethylated cap analog (e.g., m2,2,7GpppG), dimethylated symmetrical cap analogs (e.g., m7Gpppm7G), or anti reverse cap analogs (e.g., ARCA; m7,2′OmeGpppG, m7,2′dGpppG, m7,3′OmeGpppG, m7,3′dGpppG and their tetraphosphate derivatives) (Stepinski et al., 2001. RNA 7(10):1486-95). Examples of cap analogs are shown in Table 1.

TABLE 1
Cap analogs (D1 and D2 denote counterpart diastereoisomers)

| Triphosphate cap analog | Tetraphosphate cap analog |
|---|---|
| m⁷Gp₃G | m⁷Gp₄G |
| m₂^{7,3′-O}Gp₃G | b⁷Gp₄G |
| b⁷Gp₃G | b⁷m^{3′-O}Gp₄G |
| e⁷Gp₃G | m₂^{2,7}Gp₄G |
| m₂^{2,7}Gp₃G | m₃^{2,2,7}Gp₄G |
| m₃^{2,2,7}Gp₃G | b⁷m²Gp₄G |
| m⁷Gp₃2′dG | m7Gp⁴m⁷G |
| m⁷Gp₃m^{2′-O}G | |
| m⁷Gp₃m⁷G | |
| m₂^{7,2′-O}Gp₃G | |
| m₂^{7,2′-O}GpppₛG (D1) | |
| m₂^{7,2′-O}GpppₛG (D2) | |
| m₂^{7,2′-O}GppₛpG (D1) | |
| m₂^{7,2′-O}GppₛpG (D2) | |
| m₂^{7,2′-O}GpₛppG (D1) | |
| m₂^{7,2′-O}GpₛppG (D2) | |

Further cap analogs have been described previously (U.S. Pat. No. 7,074,596, WO 2008/016473, WO 2008/157688, WO 2009/149253, WO 2011/015347, and WO 2013/059475). The synthesis of N⁷-(4-chlorophenoxyethyl) substituted dinucleotide cap analogs has been described recently (Kore et al., 2013. Bioorg. Med. Chem. 21(15):4570-4).

Particularly preferred cap analogs are G[5′]ppp[5′]G, m⁷G[5′]ppp[5′]G, m₃^{2,2,7}G[5′]ppp[5′]G, m₂^{7,3′-O}G[5′]ppp[5′]G (3′-ARCA), m₂^{7,2′-O}GpppG (2′-ARCA), m₂^{72′-O}GppspG D1 (β-S-ARCA D1) and m₂^{7,2′-O}GppspG D2 (β-S-ARCA D2).

Purification/purifying: The terms “purification”, “purified” or “purifying” are intended to mean that the target RNA is separated and/or isolated from the by-products and the components of the RNA in vitro transcription reaction present in the sample comprising the target RNA after the RNA in vitro transcription reaction. Thus, after purification the purified target RNA sample has a higher purity than the target RNA-containing sample prior to purification, i.e. the amount of by-products and the components of the RNA in vitro transcription reaction is lower than in the sample after transcription, but before purification. Undesired constituents of RNA-containing samples which therefore need to be separated may in particular be by-products of the RNA in vitro transcription reaction, or also excessively long transcripts if plasmids are not completely linearised. In addition, components of the RNA in vitro transcription reaction mixture, such as enzymes, for example RNases and polymerases, and nucleotides may be separated from the target RNA in the purification step.

After the purification step, the target RNA has a higher purity than before the purification step, but may still contain by-products which may be detected by the method of the present invention. The degree of purity after the purification step may be more than 70% or 75%, in particular more than 80% or 85%, very particularly more than 90% or 95% and most favorably 99% or more. The degree of purity may for example be determined by an analytical HPLC as described herein, wherein the percentage provided above corresponds to the ratio between the area of the peak for the target RNA and the total area of all peaks representing the by-products.

HPLC: HPLC is the common abbreviation of the term “high performance liquid chromatography”. In the HPLC process a pressurized liquid solvent containing the sample mixture is passed through a column filled with a solid adsorbent material leading to the interaction of components of the sample with the adsorbent material. Since different components interact differently with the adsorbent material, this leads to the separation of the components as they flow out of the column. The operational pressure in HPLC process is typically between 50 and 350 bar. The term HPLC includes reversed phase HPLC (RP-HPLC), size exclusion chromatography, gel filtration, affinity chromatography, hydrophobic interaction chromatography or ion pair chromatography, wherein reversed phase HPLC is preferred.

Reversed phase HPLC: Reversed phase HPLC uses a non-polar stationary phase and a moderately polar mobile phase and therefore works with hydrophobic interactions which result from repulsive forces between a relatively polar solvent, the relatively non-polar analyte, and the non-polar stationary phase (reversed phase principle). The retention time on the column is therefore longer for molecules which are more non-polar in nature, allowing polar molecules to elute more readily. The retention time is increased by the addition of polar solvent to the mobile phase and decreased by the addition of more hydrophobic solvent.

The characteristics of the specific RNA molecule as an analyte may play an important role in its retention characteristics. In general, an analyte having more apolar functional groups results in a longer retention time because it increases the molecule's hydrophobicity and therefore the interaction with the non-polar stationary phase. Very large molecules, however, can result in incomplete interaction between the large analyte surface and the alkyl chain. Retention time increases with hydrophobic surface area which is roughly inversely proportional to solute size. Branched chain compounds elute more rapidly than their corresponding isomers because the overall surface area is decreased.

Ion-pair, reversed-phase HPLC: Ion-pair, reversed-phase HPLC is a specific form of reversed-phase HPLC in which an ion with a lipophilic residue and positive charge such as an alkylammonium salt, e.g. triethylammonium acetate, is added to the mobile phase as counterion for the negatively charged RNA. When used with common hydrophobic HPLC phases in the reversed-phase mode, ion pair reagents can be used to selectively increase the retention of the RNA.

Ribozyme: A ribozyme is a catalytic nucleic acid molecule which is an RNA molecule capable of catalyzing reactions including, but not limited to, site-specific cleavage of other nucleic acid molecules such as RNA molecules. The term ribozyme is used interchangeably with phrases such as catalytic RNA, enzymatic RNA, or RNA enzyme.

Ribozymes are broadly grouped into two classes based on their size and reaction mechanisms: large and small ribozymes. The first group consists of the self-splicing group I and group II introns as well as the RNA component of RNase P, whereas the latter group includes the hammerhead, hairpin, hepatitis delta ribozymes and varkud satellite (VS) RNA as well as artificially selected nucleic acids. Large ribozymes consist of several hundred up to 3000 nucleotides and they generate reaction products with a free 3′-hydroxyl and 5′-phosphate group. In contrast, small catalytically active nucleic acids from 30 to −150 nucleotides in length generate products with a 2′-3′-cyclic phosphate and a 5′-hydroxyl group (Schubert and Kurreck (2004) Curr. Drug Targets 5(8):667-681).

3′ terminus of the target RNA: The 3′ terminus of the target RNA is a region comprising nucleotides from the 3′ terminal part of the target RNA. Hence, the 3′ terminus has the same sequence as the corresponding part of the target RNA. The 3′ terminus comprises at least a part of the poly(A) sequence which is the most 3′ part of the target RNA and may additionally comprise part of the open reading frame and/or optionally the 3′ UTR (if it is encoded by the DNA template).

### DETAILED DESCRIPTION OF THE INVENTION

As discussed above, the present invention is based on the finding that by-products of RNA in vitro transcription can be detected and analyzed by HPLC.

Accordingly, the present invention relates to a method for detecting by-products of in vitro transcription in a sample comprising an in vitro transcribed target RNA, the method comprising the steps of:
a) preparing a sample comprising a target RNA by in vitro transcription;
b) purifying the target RNA, thereby providing a purified target RNA sample;
c) detecting the by-products in the purified target RNA sample by HPLC.

In one embodiment the method does not comprise a step of treating the target RNA with a ribozyme, in particular a ribozyme which is designed to cleave within the target RNA. Hence, the method does not involve a step of intentionally cleaving the target RNA to create smaller fragments, but the by-products which are detected and optionally analyzed by the method of the present invention are created unintentionally during the process of RNA in vitro transcription without requiring any step of treating the target RNA to create smaller RNA fragments.

In this context it is particularly preferred that the present invention does not relate to a method for analyzing an RNA molecule having a cleavage site for a catalytic nucleic acid molecule or a method for analyzing a population of RNA molecules, wherein the population comprises at least one RNA molecule that has a cleavage site for a catalytic nucleic acid molecule, comprising a step of determining a physical property of the at least one RNA molecule having a cleavage site by analyzing the at least one 5′ terminal RNA fragment or the 3′ terminal RNA fragment and/or the at least one optional central RNA fragment obtained by cleaving the RNA molecule with the catalytic nucleic acid molecule into a 5′ terminal RNA fragment and at least one 3′ RNA fragment and optionally into at least one central RNA fragment by contacting the RNA molecule with the catalytic nucleic acid molecule under conditions allowing the cleavage of the RNA molecule.

Furthermore, it is particularly preferred that the present invention does not relate to a method for determining the presence of a CAP structure in an RNA molecule having a cleavage site for a catalytic nucleic acid molecule, a method for determining the capping degree of a population of RNA molecules having a cleavage site for a catalytic nucleic acid molecule, a method for determining the orientation of the cap structure in a capped RNA molecule having a cleavage site for a catalytic nucleic acid molecule and a method for determining relative amounts of correctly capped RNA molecules and reverse-capped RNA molecules in a population of RNA molecules, wherein the population comprises correctly capped and/or reverse-capped RNA molecules that have a cleavage site for a catalytic nucleic acid molecule.

In one embodiment the by-products comprise at least one nucleic acid molecule, preferably at least two nucleic acid molecules with different length. This at least one nucleic acid molecule with different length is present in addition to the full-length target RNA so that in the detection step by HPLC at least two peaks appear in the chromatogram, wherein the highest peak is the peak of the full-length target RNA and the lower peak corresponds to the by-product which is present in the sample. The length of any short by-product is between 5 to 500 nucleotides, preferably between 5 and 400 or between 5 and 300 nucleotides, more preferably between 5 and 250 nucleotides and most preferably between 5 and 200 nucleotides. In particular, the by-products which are homooligomers of nucleotides have a length of 5 to 15 nucleotides, preferably of 5 to 10 nucleotides, more preferably of 5 to 7 nucleotides and most preferably of 5 nucleotides. The by-products which are short single-stranded RNAs produced by premature termination of transcription have a length of 20 to 500 nucleotides, preferably of 50 to 400 nucleotides, more preferably of 80 to 300 nucleotides and most preferably of 100 to 250 nucleotides. The length of the by-products can be determined by HPLC, preferably by reversed-phase HPLC and more preferably by ion-pair reversed-phase HPLC, wherein smaller molecules elute earlier than larger molecules. An alternative method for determining the length of the by-products is capillary gel electrophoresis.

The by-products of the present invention do not have a predetermined size, i.e. the size of the by-products only becomes apparent by the method of the present invention and cannot be predicted based on cleavage sites present within the target RNA.

The target RNA is preferably longer than any of the by-products and may have a size of up to about 15000 nucleotides, preferably of 500 to 10000 nucleotides, more preferably of 700 to 8000 nucleotides, even more preferably 800 to 5000 nucleotides and most preferably 900 to 2000 nucleotides.

The sample comprising the target RNA may be denatured before it is purified according to step b) of the method of the present invention. By denaturing the sample comprising the target RNA any intramolecular double strands formed between two RNA strands or between an RNA strand and a DNA strand are disrupted so that in the following steps only single-stranded nucleic acid molecules are present in the sample. The sample comprising the target RNA may be denatured by heating the sample comprising the in vitro transcribed RNA to a temperature at which the hydrogen bonds between the two strands are broken, such as a temperature of 90° C. Alternatively or additionally, the sample comprising the target RNA may be treated with a denaturing agent such as urea. Preferably, within the method of the present invention the target RNA is not treated with urea.

### Purification of the Target RNA (Step b) of the Method of the Invention)

In step b) of the method of the present invention the target RNA is purified by any suitable method. The method for purifying the target RNA is chosen so that the reagents (such as nucleotides and RNA polymerase) and by-products of the RNA in vitro transcription reaction are removed from the sample as completely as possible.

Suitable purification methods include alcoholic precipitation, LiCl precipitation, HPLC such as reversed-phase HPLC, anion exchange chromatography, hydroxyapatite chromatography and core bead chromatography, tangential flow filtration, gel filtration chromatography, silica membranes such as, for example, PureYield™ RNA Midiprep System of Promega and affinity chromatography (either if a tag is attached to the target RNA or via the poly(A) stretch). Preferably, the target RNA is purified using reversed-phase HPLC. Also preferably, the step of purifying the target RNA does not involve a step of spin filtration.

The HPLC for purifying the RNA is preferably performed on a preparative scale in which relatively large quantities of RNA are purified. Such relatively large quantities are for example quantities of 0.5 mg or more, in particular 1.0 mg to 1000 mg or more, very particularly approximately 1.5 mg or more, upscaling even to the kg range being possible. The above statements are to be understood to mean that these quantities relate to a single HPLC run. If a plurality of HPLC runs is performed, the quantity increases in direct proportion to the number of HPLC runs.

A particularly preferred method for purifying the target RNA is disclosed in WO 2008/077592 A1 and involves a reversed-phase HPLC using a porous reversed phase as stationary phase.

In general, any material known to be used as reverse phase stationary phase, in particular any polymeric material may be used for the inventive method, if that material can be provided in porous form. The stationary phase may be composed of organic and/or inorganic material. Examples for polymers to be used for the purification step of the present invention are (non-alkylated) polystyrenes, (non-alkylated) polystyrenedivinylbenzenes, silica gel, silica gel modified with non-polar residues, particularly silica gel modified with alkyl containing residues, more preferably with butyl-, octyl and/or octadecyl containing residues, silica gel modified with phenylic residues, polymethacrylates, etc.

In a particularly preferred embodiment, the material for the reversed phase is a porous polystyrene polymer, a (non-alkylated) porous polystyrenedivinylbenzene polymer, porous silica gel, porous silica gel modified with non-polar residues, particularly porous silica gel modified with alkyl containing residues, more preferably with butyl-, octyl and/or octadecyl containing residues, porous silica gel modified with phenylic residues, porous polymethacrylates, wherein in particular a porous polystyrene polymer or a non-alkylated (porous) polystyrenedivinylbenzene may be used.

A non-alkylated porous polystyrenedivinylbenzene which is very particularly preferred for the purification step of the method according to the invention is one which, without being limited thereto, may have a particle size of 8.0±1.5 μm, in particular 8.0±0.5 μm, and a pore size of 1000-1500 Å, in particular 1000-1200 Å or 3500-4500 Å.

The stationary phase is conventionally located in a column. V2A steel is conventionally used as the material for the column, but other materials may also be used for the column provided they are suitable for the conditions prevailing during HPLC. Conventionally the column is straight. It is favourable for the HPLC column to have a length of 5 cm to 100 cm and a diameter of 4 mm to 25 mm. Columns used for the purification step of the method of the invention may in particular have the following dimensions: 50 mm long and 7.5 mm in diameter or 50 mm long and 4.6 mm in diameter, or 50 mm long and 10 mm in diameter or any other dimension with regard to length and diameter, which is suitable for preparative recovery of RNA, even lengths of several metres and also larger diameters being feasible in the case of upscaling.

The HPLC is preferably performed as ion-pair, reversed phase HPLC as defined above.

In a preferred embodiment, a mixture of an aqueous solvent and an organic solvent is used as the mobile phase for eluting the RNA. Preferably, the buffer used as the aqueous solvent has a pH of 6.0-8.0, for example of about 7, for example 7.0. More preferably the buffer is triethylammonium acetate which preferably has a concentration of 0.02 M to 0.5 M, more preferably of 0.08 M to 0.12 M. Most preferably, an 0.1 M triethylammonium acetate buffer is used, which also acts as a counterion to the RNA in the ion pair method.

In a preferred embodiment, the organic solvent which is used in the mobile phase is selected from acetonitrile, methanol, ethanol, 1-propanol, 2-propanol and acetone or a mixture thereof. More preferably it is acetonitrile.

In a particularly preferred embodiment, the mobile phase is a mixture of 0.1 M triethylammonium acetate, pH 7, and acetonitrile.

Preferably, the mobile phase contains 5.0 vol. % to 25.0 vol. % organic solvent, relative to the mobile phase, and for this to be made up to 100 vol. % with the aqueous solvent. Typically, in the event of gradient separation, the proportion of organic solvent is increased, in particular by at least 10%, more preferably by at least 50% and most preferably by at least 100%, optionally by at least 200%, relative to the initial vol. % in the mobile phase. In a preferred embodiment, the proportion of organic solvent in the mobile phase amounts in the course of HPLC separation to 3 to 9, preferably 4 to 7.5, in particular 5.0 vol. %, in each case relative to the mobile phase. More preferably, the proportion of organic solvent in the mobile phase is increased in the course of HPLC separation from 3 to 9, in particular 5.0 vol. % to up to 20.0 vol. %, in each case relative to the mobile phase. Still more preferably, the method is performed in such a way that the proportion of organic solvent in the mobile phase is increased in the course of HPLC separation from 6.5 to 8.5, in particular 7.5 vol. %, to up to 17.5 vol. %, in each case relative to the mobile phase.

Even more preferably the mobile phase contains 7.5 vol. % to 17.5 vol. % organic solvent, relative to the mobile phase, and for this to be made up to 100 vol. % with the aqueous buffered solvent.

Elution may proceed isocratically or by means of gradient separation. In isocratic separation, elution of the RNA proceeds with a single eluent or a constant mixture of a plurality of eluents, wherein the solvents described above in detail may be used as eluent.

In a preferred embodiment, gradient separation is performed wherein the composition of the eluent is varied by means of a gradient program. The equipment necessary for gradient separation is known to a person skilled in the art. Gradient elution may here proceed either on the low pressure side by mixing chambers or on the high pressure side by further pumps.

Preferably, the proportion of organic solvent, as described above, is increased relative to the aqueous solvent during gradient separation. The above-described agents may here be used as the aqueous solvent and the likewise above-described agents may be used as the organic solvent. For example, the proportion of organic solvent in the mobile phase may be increased in the course of HPLC separation from 5.0 vol. % to 20.0 vol. %, in each case relative to the mobile phase. In particular, the proportion of organic solvent in the mobile phase may be increased in the course of HPLC separation from 7.5 vol. % to 17.5 vol. %, in particular 9.5 to 14.5 vol. %, in each case relative to the mobile phase.

The following gradient program has proven particularly favourable for the purification of RNA:

Eluent A: 0.1 M triethylammonium acetate, pH 7
Eluent B: 0.1 M triethylammonium acetate, pH 7, with 25 vol. % acetonitrile

### Eluent Composition:

start: 62% A and 38% B (1 st to 3rd minute)
increase to 58% B (1.67% increase in B per minute), (3rd-15th minute)
100% B (15th to 20th minute)

Another example of a gradient program is described below, the same eluent A and B being used:

### Eluent Composition:

starting level: 62% A and 38% B (1 st-3rd min)
separation range I: gradient 38%-49.5% B (5.75% increase in B/min) (3rd-5th min)
separation range II: gradient 49.5%-57% B (0.83% increase in B/min) (5th-14th min)
rinsing range: 100% B (15th-20th min)

It is preferred to use purified solvent for HPLC. Such purified solvents are commercially obtainable. They may additionally also be filtered through a 1 to 5 μm microfilter, which is generally mounted in the system upstream of the pump. It is additionally preferred for all the solvents to be degassed prior to use, since otherwise gas bubbles occur in most pumps. If air bubbles occur in the solvent, they may interfere not only with separation but also with the continuous monitoring of outflow in the detector. The solvents may be degassed by heating, by vigorous stirring with a magnetic stirrer, by brief evacuation, by ultrasonication or by passing a small stream of helium through the solvent storage vessel.

The flow rate of the eluent is selected such that good separation of the RNA from the other constituents contained in the sample to be investigated takes place. The eluent flow rate may amount to from 1 ml/min to several litres per minute (in the case of upscaling), in particular about 1 to 1000 ml/min, more preferably 5 ml to 500 ml/min, even more preferably more than 100 ml/min, depending on the type and scope of the upscaling. This flow rate may be established and regulated by the pump.

The HPLC is preferably performed under denaturing conditions, such as an increased temperature. Suitable temperature conditions include a temperature of at least 70° C., preferably of at least 75° C., more preferably of about 78° C. By using denaturing conditions any intramolecular double strands formed between two RNA strands or between an RNA strand and a DNA strand are disrupted so that only single-stranded nucleic acid molecules are present in the sample.

Detection proceeds preferably with a UV detector at 254 nm, wherein a reference measurement may be made at 600 nm. However, any other detection method may alternatively be used, with which the RNA may be detected.

For preparative purification of the RNA, it is advisable to collect the RNA-containing eluted solvent quantities. In this respect, it is preferred to carry out this collection in such a way that the eluted solvent is collected in individual separated fractions. This may take place for example with a fraction collector. In this way, the high-purity RNA-containing fractions may be separated from other RNA-containing fractions which still contain undesired impurities, albeit in very small quantities. The individual fractions may be collected for example over 1 minute.

The method according to the invention is preferably performed under completely denaturing conditions. This may proceed for example in that sample application takes place at a temperature of 4-12° C., the HPLC method otherwise proceeding at a higher temperature, preferably at 70° C. or more, particularly preferably at 75° C. or more, in particular up to 82° C., and very particularly preferably at about 78° C.

Sample application may be performed with two methods, stop-flow injection or loop injection. For stop-flow injection a microsyringe is used which is able to withstand the high pressure applied in HPLC. The sample is injected through a septum in an inlet valve either directly onto the column packing or onto a small drop of inert material immediately over the packing. The system may in this case be under elevated pressure, or the pump may be turned off prior to injection, which is then performed when the pressure has fallen to close to the normal value. In the case of loop injection, a loop injector is used to introduce the sample. This consists of a tubular loop, into which the sample is inserted. By means of a suitable rotary valve, the stationary phase is then conveyed out of the pump through the loop, whose outlet leads directly into the column. The sample is entrained in this way by the stationary phase into the column, without solvent flow to the pump being interrupted.

Detection of by-Products (Step c) of the Method of the Invention)

After the target RNA has been purified to provide a purified target RNA sample, all or part of the purified target RNA sample is analyzed by HPLC to detect by-products. Preferably, only a part of the purified target RNA sample, such as 20% or 15%, preferably 10% or 8%, more preferably 5% or 2% and most preferably 1% of the volume of the purified target RNA sample or less is used for the HPLC analysis to detect by-products.

The HPLC analysis of step c) of the method of the present invention is therefore preferably performed at an analytical scale. In an analytical HPLC method, a quantity of RNA such as 8 ng to 1000 ng or 20 to 100 μg is introduced for a single HPLC run. If a plurality of HPLC runs is performed, the quantity increases in direct proportion to the number of HPLC runs.

The remainder of the purified target RNA sample can be further processed to the final RNA product, such as a RNA product for administration to a patient, if the HPLC analysis according to step c) of the method of the invention indicates that the amount of by-products is within a range which is acceptable for a final RNA product.

Stationary phases for use in the HPLC analysis are known in the art. Preferably, the stationary phase is selected from the group consisting of a porous polystyrene, a porous non-alkylated polystyrene, a polystyrenedivinylbenzene, a porous non-alkylated polystyrenedivinylbenzene, a porous silica gel, a porous silica gel modified with non-polar residues, a porous silica gel modified with carbon chains, selected from butyl-, octyl and/or octadecyl carbon chains, a porous silica gel modified with phenylic residues, and a porous polymethacrylate. The stationary phase used for step c) of the method of the present invention is preferably a porous silica gel modified with carbon chains, preferably an octadecyl carbon chain.

More preferably the porous silica gel is prepared from tetraethoxysilane and bis(triethoxysilyl)ethane which are even more preferably used in a 4:1 mole ratio. Most preferably, the porous silica gel is prepared from tetraethoxysilane and bis(triethoxysilyl)ethane which are used in a 4:1 mole ratio and the porous silica gel is modified with an octadecyl carbon chain. Such porous silica gel is commercially available, e.g. XBRIDGE™ OST C₁₈ from Waters or AQUITY UPLC OST C18 from Waters, and is described in more detail in Wyndham et al. (2003) Anal. Chem. 75(24):6781-8 and WO 2003/014450.

The silica gel may have a particle size of 0.5 to 5 μm, preferably of 0.7 to 4 μm, more preferably of 1 to 3 μm, even more preferably of 1.5 to 2 μm and most preferably of 1.7 μm. The pore size of the porous silica gel may be 50 to 300 Å, preferably 70 to 250 Å, more preferably 100 to 200 Å, even more preferably 120 to 170 Å and most preferably it is 135 Å.

Hence, in a most preferred embodiment the stationary phase is a porous silica gel prepared from tetraethoxysilane and bis(triethoxysilyl)ethane which are used in a 4:1 mole ratio, wherein the porous silica gel is modified with an octadecyl carbon chain and has a pore size of 135 Å and a particle size of 1.7 μm.

The stationary phase is conventionally located in a column. V2A steel is conventionally used as the material for the column, but other materials may also be used for the column provided they are suitable for the conditions prevailing during HPLC. Conventionally the column is straight. It is preferred for the HPLC column to have a length of 5 cm to 100 cm and a diameter of 0.5 mm to 10 mm. Columns used for the method according to the invention may in particular have the following dimensions: 50 mm long and 4.6 mm in diameter or 50 mm long and 2.1 mm in diameter.

The HPLC is preferably performed as ion-pair, reversed phase HPLC as defined above.

In a preferred embodiment, a mixture of an aqueous solvent, preferably a buffer, and an organic solvent is used as the mobile phase for eluting the RNA.

Preferably, the buffer used as the aqueous solvent has a pH of 6.0-8.0, for example of about 7, for example 7.0.

The buffer may be selected from the group consisting of triethylammonium acetate, trifluoroacetic acid, acetic acid, formic acid, acetate buffer, phosphate buffer, tetrabutylammonium bisulfate, tetrabutylammonium bromide and tetrabutylammonium chloride.

Preferably the buffer is triethylammonium acetate which preferably has a concentration of 0.02 M to 0.5 M, more preferably of 0.08 M to 0.12 M. Most preferably, an about 0.1 M triethylammonium acetate buffer is used, which also acts as a counterion to the RNA in the ion pair method.

In a preferred embodiment, the organic solvent which is used in the mobile phase is selected from acetonitrile, methanol, ethanol, 1-propanol, 2-propanol, hexafluoroisopropanol and acetone or a mixture thereof. More preferably it is acetonitrile.

In a particularly preferred embodiment, the mobile phase is a mixture of 0.1 M triethylammonium acetate, pH 7.3, and acetonitrile.

Preferably, the mobile phase which is applied to the HPLC column contains 3.0 vol. % to 5.0 vol. % organic solvent, preferably 3.25 to 4.0 vol. % and most preferably 3.75 vol. % organic solvent, preferably acetonitrile, relative to the mobile phase, and is made up to 100 vol. % with the aqueous solvent.

Elution may proceed isocratically or by means of gradient separation. In isocratic separation, elution of the RNA proceeds with a single eluent or a constant mixture of a plurality of eluents, wherein the solvents described above in detail may be used as eluent. In a preferred embodiment, gradient separation is performed wherein the composition of the eluent is varied by means of a gradient program. The equipment necessary for gradient separation is known to a person skilled in the art. Gradient elution may here proceed either on the low pressure side by mixing chambers or on the high pressure side by further pumps.

Typically, in the event of gradient separation, the proportion of organic solvent, in particular of acetonitrile, is increased in the course of HPLC separation in one or more steps. Preferably, the proportion of the organic solvent, in particular of acetonitrile, is increased in the course of HPLC separation in two steps, more preferably the proportion of the organic solvent, in particular of acetonitrile, is increased in the course of HPLC separation in three steps.

In one embodiment, the proportion of the organic solvent, in particular of acetonitrile, is increased in the course of HPLC separation from 3.5 vol. % to 100 vol. %, relative to the mobile phase, and is made up to 100 vol. % with the aqueous solvent. After increasing the proportion of the organic solvent, in particular of acetonitrile, the proportion may again be decreased, e.g. from 100 vol. % to 3.5 vol. %, the rest being the aqueous solvent.

The following gradient program has proven particularly useful in the HPLC method for detecting by-products:

Eluent A: 0.1 M triethylammonium acetate, pH 6.8
Eluent B: 0.1 M triethylammonium acetate, pH 7.3, with 25 vol. % acetonitrile

### Eluent Composition:

start: 86% A and 14% B (1 st to 3rd minute)
increase to 19% B over 2 minutes
increase to 21% B over 9 minutes, then holding at 21% B for one minute
increase to 100% B over 5 minutes, then holding at 100% B for 3.5 minutes
decrease to 14% over 1.5 minutes

It is preferred to use purified solvent for HPLC. Such purified solvents are commercially obtainable. They may additionally also be filtered through a 1 to 5 μm microfilter, which is generally mounted in the system upstream of the pump. It is additionally preferred for all the solvents to be degassed prior to use, since otherwise gas bubbles occur in most pumps. If air bubbles occur in the solvent, they may interfere not only with separation but also with the continuous monitoring of outflow in the detector. The solvents may be degassed by heating, by vigorous stirring with a magnetic stirrer, by brief evacuation, by ultrasonication or by passing a small stream of helium through the solvent storage vessel.

The flow rate of the eluent is selected such that good separation of the RNA from the other constituents contained in the sample to be investigated takes place. The eluent flow rate is between 50 and 80 ml/min, preferably between 55 and 75 ml/min, more preferably between 60 and 70 ml/min and most preferably it is 0.65 ml/min. This flow rate may be established and regulated by the pump.

Detection proceeds preferably with a UV detector at 254 nm, wherein a reference measurement may be made at 600 nm. However, any other detection method may alternatively be used, with which the RNA may be detected.

The method according to the invention is preferably performed at elevated temperature. For example, the sample comprising the purified target RNA or a fraction thereof may be applied to the column at a temperature of 4-12° C., and the subsequent steps may be performed at a higher temperature, preferably at 50° C. or more, particularly preferably at 55° C. or more and most preferably at about 60° C.

Sample application may be performed with two methods, stop-flow injection or loop injection. For stop-flow injection a microsyringe is used which is able to withstand the high pressure applied in HPLC. The sample is injected through a septum in an inlet valve either directly onto the column packing or onto a small drop of inert material immediately over the packing. The system may in this case be under elevated pressure, or the pump may be turned off prior to injection, which is then performed when the pressure has fallen to close to the normal value. In the case of loop injection, a loop injector is used to introduce the sample. This consists of a tubular loop, into which the sample is inserted. By means of a suitable rotary valve, the stationary phase is then conveyed out of the pump through the loop, whose outlet leads directly into the column. The sample is entrained in this way by the stationary phase into the column, without solvent flow to the pump being interrupted.

Within the method of the present invention the step of detecting by-products may also comprise determining the amount of each by-product or the total amount of all by-products present in the sample.

The term “amount of each by-product”, as used herein, means the amount of a specific by-product present within a sample. It can be determined by calculating the area of the peak corresponding to said specific by-product and relating this area to the area of the peak of the target RNA.

The term “total amount of all by-products”, as used herein, means the total amount of all by-products present within a sample. It can be determined by calculating the area of all peaks representing by-products and relating this area to the area of the peak of the target RNA.

After detecting the by-products in the purified target RNA sample by HPLC, said by-products may be isolated and/or characterized. The by-products may be isolated by collecting the RNA-containing eluted solvent quantities. In this respect, it is preferred to carry out this collection in such a way that the eluted solvent is collected in individual separated fractions. This may take place for example with a fraction collector. In this way, the by-product-containing fractions may be separated from the fractions containing the target RNA. Further, if the HPLC analysis shows more than one by-product peak, each of the by-products corresponding to one of the peaks may be collected separately, allowing the separate analysis of each by-product. The individual fractions may be collected for example over 1 minute.

The by-products may be characterized by any suitable method of RNA analysis, including enzyme assays, spectroscopic methods, mass spectrometry and sequencing.

Spectroscopic methods for RNA analysis include traditional absorbance measurements at 260 nm and more sensitive fluorescence techniques using fluorescent dyes such as ethidium bromide and a fluorometer with an excitation wavelength of 302 or 546 nm (Gallagher (2011) Current Protocols in Molecular Biology. 93:A.3D.1-A.3D.14).

A mass spectrometer (MS) is a gas phase spectrometer that measures a parameter that can be translated into mass-to-charge ratio of gas phase ions. Examples of mass spectrometers are time-of-flight, magnetic sector, quadrupole filter, ion trap, ion cyclotron resonance, electrostatic sector analyser and hybrids of these. Methods for the application of MS methods to the characterization of nucleic acids are known in the art.

For example, Matrix-Assisted Laser Desorption/Ionization Mass Spectrometry (MALDI-MS) can be used to analyse oligonucleotides at the 120-mer level and below (Castleberry et al. (2008) Current Protocols in Nucleic Acid Chemistry. 33: 10.1.1-10.1.21).

Electrospray Ionization Mass Spectrometry (ESI-MS) allows the analysis of high-molecular-weight compounds through the generation of multiply charged ions in the gas phase and can be applied to molecular weight determination, sequencing and analysis of oligonucleotide mixtures (Castleberry et al. (2008) Current Protocols in Nucleic Acid Chemistry. 35: 10.2.1-10.2.19). Preferably, the mass spectrometry analysis is conducted in a quantitative manner to determine the amount of RNA.

Methods for sequencing of RNA are known in the art. A recently developed technique called RNA Sequencing (RNA-Seq) uses massively parallel sequencing to allow for example transcriptome analyses of genomes at a far higher resolution than is available with Sanger sequencing and microarray-based methods. In the RNA-Seq method, complementary DNAs (cDNAs) generated from the RNA of interest are directly sequenced using next-generation sequencing technologies. RNA-Seq has been used successfully to precisely quantify transcript levels, confirm or revise previously annotated 5′ and 3′ ends of genes, and map exon/intron boundaries (Eminaga et al. (2013) Current Protocols in Molecular Biology. 103: 4.17.1-4.17.14). Consequently, the amount of the by-products can be determined also by RNA sequencing.

The method of the present invention may be used to identify sequence motifs within the RNA which are responsible for the generation of the by-products. For example, several RNA sequences encoding the same protein, but differing in the coding sequence or the presence and/or type of 5′ or 3′ untranslated region may be produced and the amount and optionally the identity of the by-products can be determined for each of these RNA sequences by the method of the present invention, leading to the selection of a construct encoding the RNA product which construct produces the lowest amount of by-products.

The method of the present invention may also be used for the quality control of RNA produced by RNA in vitro transcription. For example, in the preparation of RNA products for pharmaceutical use a level of by-products may be defined which is acceptable for the pharmaceutical product. The method of the present invention can then be used to determine whether in a sample the total amount of the by-products is below or above the threshold. If the amount of by-products is below the threshold, the RNA product can be marketed and if the amount of the by-products is above the threshold, the RNA product has to be discarded or subjected to further purification steps.

Further, the method of the present invention may be used to compare different RNA purification conditions. For example, one or more variables in a purification protocol can be varied and then it can be determined whether the amount of by-products increases or decreases due to the variation of the purification conditions. Then those purification conditions which produce the lowest amount of by-products are selected for the preparative RNA purification.

### EXAMPLES

The Examples shown in the following are merely illustrative and shall describe the present invention in a further way. These Examples shall not be construed to limit the present invention thereto.

### Example 1: Preparation of the mRNA

### 1. Preparation of DNA Template

For the present example DNA sequences encoding PpLuc mRNA according to SEQ ID NOs: 1-3 were prepared and used for subsequent in vitro transcription reactions. The RNAs encoded by the DNA sequences had the following features:
5′ cap—GC-optimized open reading frame (ORF)—globin 3′ UTR—a stretch of 64 adenosines—a stretch of 30 cytosines (RNA R 491)
5′ cap—GC-optimized open reading frame (ORF)—globin 3′ UTR—a stretch of 64 adenosines—a stretch of 30 cytosines—a histone stem-loop sequence (RNA R 1265)
5′ cap—32L-5′-UTR—GC-optimized open reading frame (ORF)—albumin 3′ UTR—a stretch of 64 adenosines—a stretch of 30 cytosines—a histone stem-loop sequence (RNA R 2244)

The constructs were prepared by modifying the wild type coding sequence by introducing a GC-optimized sequence for stabilization, UTRs (derived from 32L4, albumin or alpha globin were introduced as indicated). The 3′-UTR was followed by a stretch of 64 adenosines (poly-A-sequence), a stretch of 30 cytosines (poly-C-sequence) and optionally a histone stem-loop sequence.

### 2. In Vitro Transcription

The DNA plasmids prepared according to section 1 were transcribed in vitro using T7 RNA polymerase.

For the production of 5′-capped RNAs using cap analog, transcription was carried out in 5.8 mM m7G(5″)ppp(5′)G Cap analog, 4 mM ATP, 4 mM CTP, 4 mM UTP, and 1.45 mM GTP (all Thermo Fisher Scientific). Subsequently the mRNA was purified using HPLC using a porous reversed phase as stationary phase (described in detail in WO2008/077592A1).

### Example 2: HPLC Determination of Short RNA by-Products

Analysis was performed via ion-pair, reversed-phase chromatography on a Dionex Parallel-HPLC U3000 CV-P-1247, equipped with analytical pump (DPG-3600SD), column oven (TCC-3000SD) and UV/Vis-4-channel-detectors (2×VWD-3400RS) with analytical SST measuring cell (11 μL, 10 mm, for VWD-3×00 detector). An AQUITY UPLC OST C18 column (2.1×50 mm, 1.7 μm particle size; Waters Corporation, Milford, Mass., USA) was used. Column temperature was set to 60° C. Buffer A contained 0.1 M triethylammonium acetate (TEAA), pH 6.8, buffer B 0.1 M TEAA, pH 7.3, 25% acetonitrile. The column was equilibrated with 14% buffer B.

For sample preparation, HPLC equilibration buffer (86% buffer A, 14% buffer B) was added to the RNA to obtain a final volume of 1700 μl.

1650 μl of the RNA solution were loaded using a SEMIPREP-Autosampler (WPS-3000SL, Dionex) and run with a stepped gradient beginning with 14% buffer B for 3 minutes, increasing to 19% buffer B over 2 minutes, to 21% buffer B over 9 minutes. 21% buffer B was held for 1 minute, then increased to 100% B over 5 minutes, held for 3.5 minutes, then decreased to 14% buffer B over 1.5 minutes.

Signal integration was done using Chromeleon software 6.80 SR11 Build 3161 (Dionex).

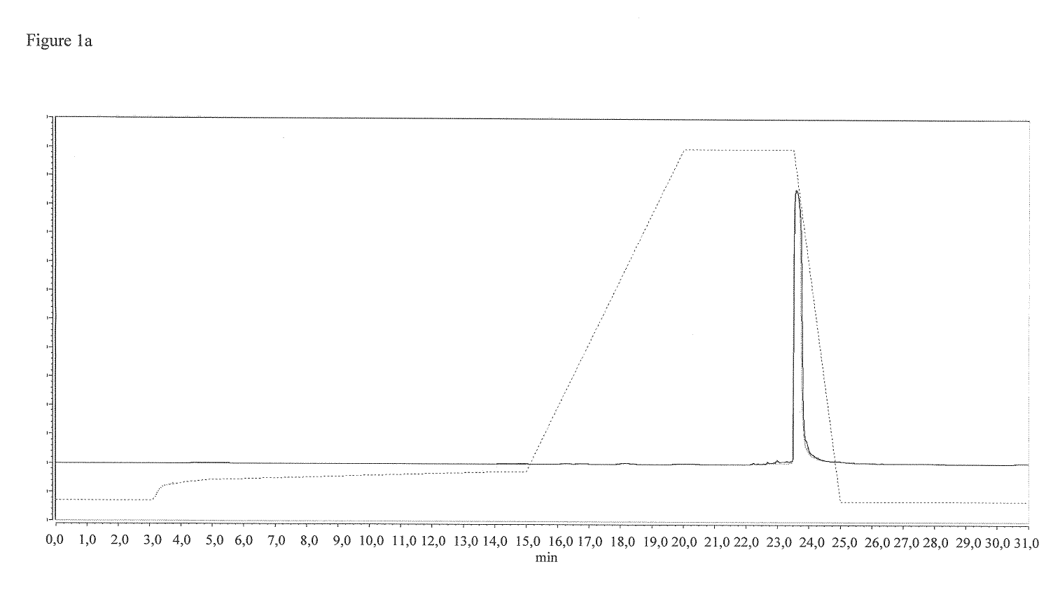

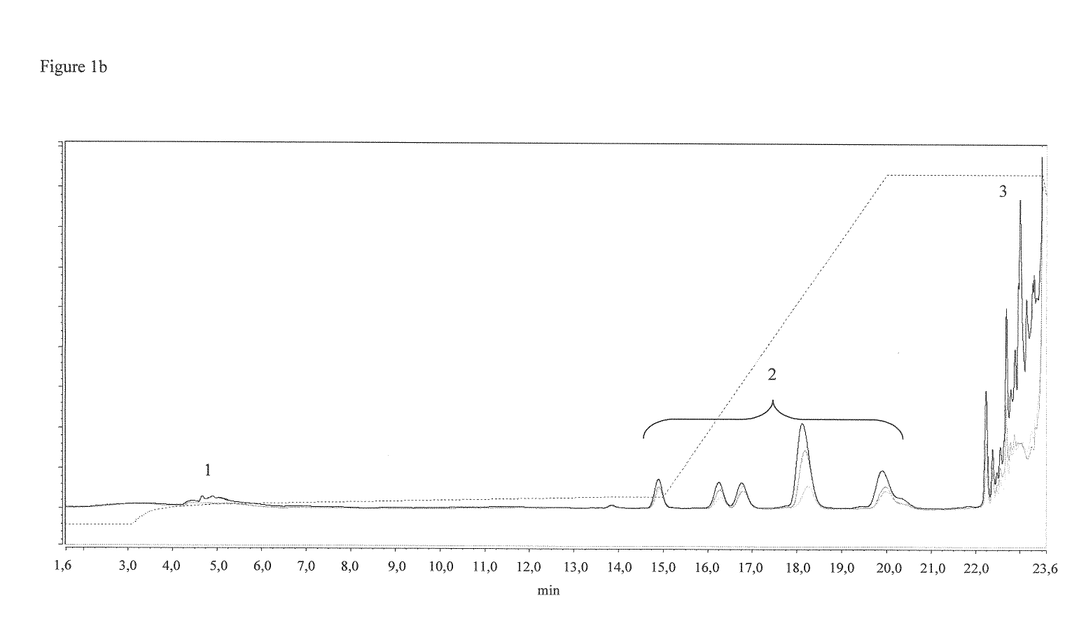

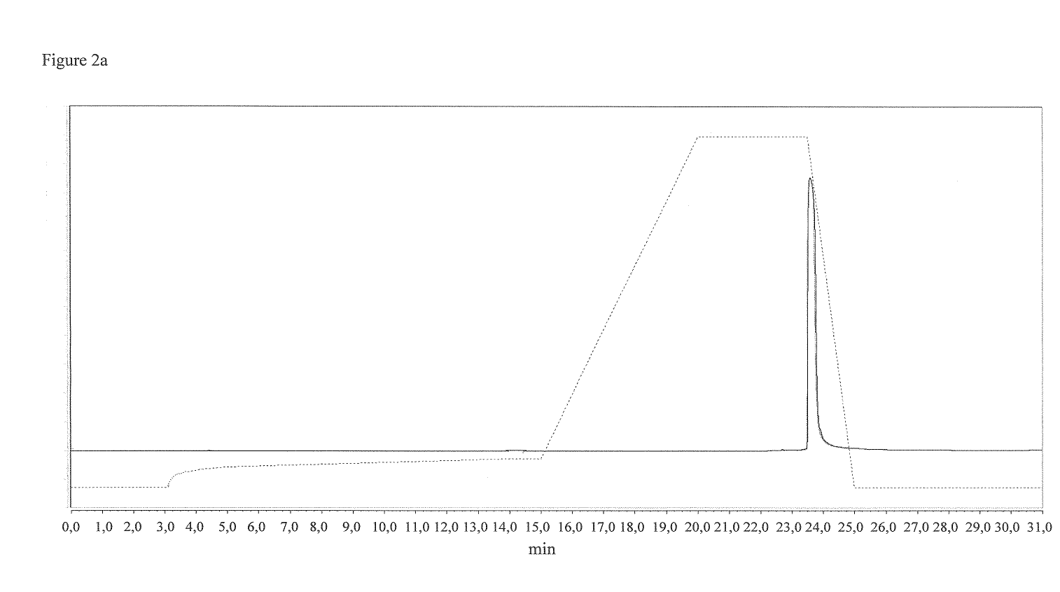

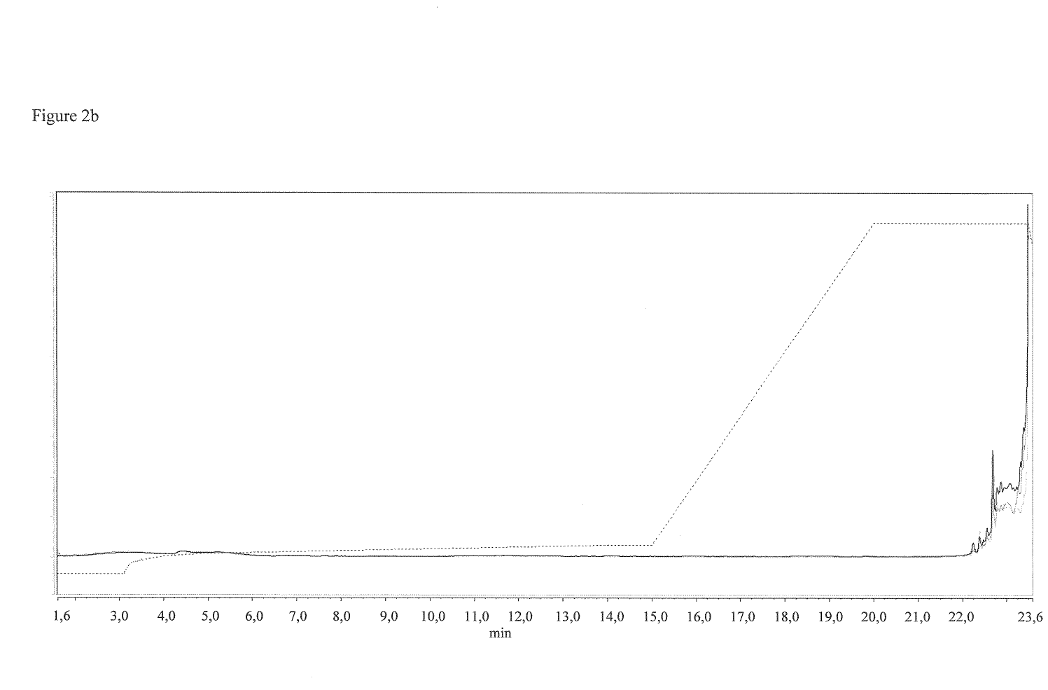

FIG. 1 shows that by applying a standard preparative HPLC method several peaks representing by-products of the RNA in vitro transcription reaction can be detected.

FIG. 2 shows that by applying an improved preparative HPLC method shorter by-products (shown as peaks (1) and (2) in FIG. 1B) could be removed, thereby increasing the quality of the mRNA product.

1. Method for detecting by-products of in vitro transcription in a sample comprising an in vitro transcribed target RNA, the method comprising the steps of:
a) preparing a sample comprising a target RNA by in vitro transcription;
b) purifying the target RNA, thereby providing a purified target RNA sample;
c) detecting the by-products in the purified target RNA sample by HPLC.

2. Method according to claim 1, wherein the method does not comprise a step of treating the target RNA with a ribozyme.

3. Method according to claim 1 or 2, wherein the by-products comprise at least two nucleic acid molecules with different length.

4. Method according to any one of the preceding claims, wherein the by-products do not comprise the 3′ terminus of the target RNA.

5. Method according to any one of the preceding claims, wherein the by-products have a length of 5 to 500 nucleotides.

6. Method according to any one of the preceding claims, wherein the by-products are homooligomers of nucleotides, short single-stranded RNAs, double-stranded RNAs and/or DNA-RNA hybrids.

7. Method according to any one of the preceding claims, wherein step b) is performed under denaturing conditions.

8. Method according to any one of the preceding claims, wherein step b) comprises a step of purifying the target RNA by HPLC.

9. Method according to any one of the preceding claims, wherein step b) comprises a step of purifying the target RNA by reversed-phase HPLC.

10. Method according to claim 8 or 9, wherein a porous reversed phase is used as stationary phase in the HPLC.

11. Method according to claim 10, wherein the porous reversed phase is a porous, non-alkylated polystyrene/divinylbenzene matrix.

12. Method according to any one of the preceding claims, wherein the HPLC in step c) is ion-pair, reversed-phase HPLC.

13. Method according to any one of the preceding claims, wherein the HPLC in step c) uses a carbon-chain bonded silica column.

14. Method according to claim 13, wherein the carbon-chain bonded silica column is an octadecyl carbon chain (C18)-bonded silica column.

15. Method according to claim 13 or 14, wherein the silica column is prepared from tetraethoxysilane and bis(triethoxysilyl)ethane.

16. Method according to claim 15, wherein tetraethoxysilane and bis(triethoxysilyl)ethane are used in a 4:1 mole ratio.

17. Method according to any one of claims 13 to 16, wherein the column has a particle size of 0.5 to 5 μm.

18. Method according to any one of claims 13 to 17, wherein the column has a pore size of 50 to 300 Å.

19. Method according to any one of the preceding claims, wherein the HPLC of step c) uses a mixture of an aqueous solvent and an organic solvent as mobile phase.

20. Method according to claim 19, wherein the aqueous solvent is a buffer.

21. Method according to claim 20, wherein the buffer is selected from the group consisting of triethylammonium acetate, trifluoroacetic acid, acetic acid, formic acid, acetate buffer, phosphate buffer, tetrabutylammonium bisulfate, tetrabutylammonium bromide and tetrabutylammonium chloride.

22. Method according to claim 20 or 21, wherein the buffer is a 0.1 M triethylammonium acetate buffer.

23. Method according to any one of claims 19 to 22, wherein the organic solvent is selected from the group consisting of acetonitrile, methanol, ethanol, 1-propanol, 2-propanol, hexafluoroisopropanol, acetone and a mixture thereof.

24. Method according to any one of claims 19 to 23, wherein the organic solvent is acetonitrile.

25. Method according to any one of claims 19 to 24, wherein at the beginning of the HPLC process the mobile phase contains 3 to 5% organic solvent, relative to the mobile phase, the rest being the aqueous solvent.

26. Method according to any one of claims 12 to 25, wherein a gradient separation proceeds.

27. Method according to claim 26, wherein the proportion of organic solvent is increased to provide the gradient.

28. Method according to claim 27, wherein the proportion of organic solvent in the mobile phase is increased in the course of HPLC separation from 3.5% to 100%.

29. Method according to any one of the preceding claims, wherein the method further comprises a step d) of isolating and/or characterizing the by-products.

30. Method according to claim 29, wherein the by-products are characterized by enzyme assays, mass spectrometry and/or sequencing.

31. Method according to any one of the preceding claims, wherein the amount of the by-products relative to the total amount of RNA is determined.

32. Use of the method according to any one of the preceding claims for identifying sequence motifs within the target RNA which are responsible for the generation of by-products.

33. Use of the method according to any one of the preceding claims for the quality control of RNA produced by in vitro transcription.

34. Use of the method according to any one of claims 1 to 31 for identifying suitable RNA purification conditions.

35. Use of the method according to any one of claims 1 to 31 for comparing RNA purification conditions.
